(19) United States
(12) Patent Application Publication
(10) Pub. No.: US 2020/0338337 A1
(43) Pub. Date: Oct. 29, 2020

(54) NON-INVASIVE INTRAORAL NEUROSTIMULATION DEVICE FOR OBSTRUCTIVE SLEEP APNEA

(71) Applicants: Stephen Lane, (US); Cami Rowan, (US); Alarmel Sira, (US); Kendra Lenz, (US); ARIZONA BOARD OF REGENTS ON BEHALF OF ARIZONA STATE UNIVERSITY, Scottsdale, AZ (US)

(72) Inventors: James Abbas, Scottsdale, AZ (US); Stephen Lane, Tempe, AZ (US); Cami Rowan, Tempe, AZ (US); Alarmel Sira, Gilbert, AZ (US); Kendra Lenz, Stevensville, MT (US)

(21) Appl. No.: 16/857,436

(22) Filed: Apr. 24, 2020

Related U.S. Application Data
(60) Provisional application No. 62/839,302, filed on Apr. 26, 2019.

(51) Int. Cl. A61N 1/05; A61N 1/36; A61B 5/08

(52) U.S. Cl. CPC A61N 1/0548; A61B 5/0826; A61N 1/36003

(57) ABSTRACT

Disclosed herein is a non-invasive intraoral neurostimulation device and method for treating obstructive sleep apnea (OSA) while improving adherence, effectiveness, and outcomes. The intraoral neurostimulation device includes a mouthpiece, at least one electrode pair having a reference electrode and a stimulating electrode supported by the mouthpiece, and a substrate positioned between a tongue of a user and the stimulating electrode. The intraoral neurostimulation device is configured to deliver non-invasive electrical neurostimulation to the motor neurons of at least one genioglossus muscle to induce a muscle contraction of the tongue, thereby eliminating the blockage and returning normal airflow to the user. In certain embodiments, the intraoral neurostimulation device is paired with a respiratory sensor to deliver stimulation synchronized with the respiratory rhythm of the user or deliver stimulation only during apneic episodes, reducing the total amount of stimulation time.

### RELATED APPLICATIONS

This application claims priority to U.S. Provisional Patent Application No. 62/839,302, filed Apr. 26, 2019, wherein the disclosure of such application is hereby incorporated by reference herein in its entirety.

### TECHNICAL FIELD

This disclosure relates to neurostimulation devices. In particular, the present disclosure relates to non-invasive intraoral neurostimulation devices.

### BACKGROUND

Obstructive sleep apnea (OSA) is a common sleep disorder characterized by upper airway obstruction. Societal costs attributable to individuals suffering from this condition include, for example, motor vehicle accidents, workplace accidents, lost productivity, healthcare expenditures, etc.

Various treatments are available, but suffer from one or more drawbacks. For example, some treatments have adherence rates that fall significantly after a year. Other treatments include continuous positive airway pressure (CPAP), which is effective, but undesirable due to bulky design, prevalence of user error, and/or discomfort, etc. Another treatment includes upper airway stimulation (UAS) devices, but such treatment requires invasive surgery and other expenses.

There is a need to enhance conventional OSA treatment methods by providing a safer, more accessible, and effective alternative solution with high adherence rates.

### SUMMARY

Disclosed herein is a non-invasive intraoral neurostimulation device to treat obstructive sleep apnea (OSA) while improving adherence, effectiveness, and outcomes. The intraoral neurostimulation device includes a mouthpiece, at least one electrode pair having a reference electrode and a stimulating electrode supported by the mouthpiece, and a substrate positioned between a tongue of a user and the stimulating electrode. The intraoral neurostimulation device is configured to deliver non-invasive electrical neurostimulation to the motor neurons of at least one genioglossus muscle to induce a muscle contraction of the tongue, thereby eliminating the blockage and returning normal airflow to the user. In certain embodiments, the intraoral neurostimulation device is paired with a respiratory sensor to deliver stimulation only during apneic episodes, thereby reducing the total amount of stimulation time. Methods of using an intraoral neurostimulation device for treating or ameliorating effects of sleep apnea are also provided.

In one aspect, the disclosure relates to an intraoral neurostimulation device, including a mouthpiece, a first electrode pair, and at least one substrate. The mouthpiece includes a dental attachment configured to be removably received by at least one tooth within a mouth of a user. The first electrode pair includes a first reference electrode and a first stimulating electrode. The first stimulating electrode is supported by the mouthpiece and is configured to be positioned between at least one genioglossus muscle and a tongue of the user. The at least one substrate is arranged between the first stimulating electrode and the tongue of the user. The first stimulating electrode is configured to transmit a first electrical signal to provide non-invasive electrical stimulation to the at least one genioglossus muscle of the user.

In certain embodiments, the at least one tooth includes at least one bottom tooth of the user.

In certain embodiments, the mouthpiece includes the at least one substrate including a first tab integrally extending from the dental attachment. The first stimulating electrode is mounted to a bottom of the first tab.

In certain embodiments, the first reference electrode is configured to be in conductive electrical communication with a jaw of the user via the mouth of the user.

In certain embodiments, the first reference electrode is configured to be in conductive electrical communication with skin of the user.

In certain embodiments, the intraoral neurostimulation device further includes a first waveform generator in electrical communication with the first electrode pair and configured to deliver a first biphasic waveform to the first electrode pair.

In certain embodiments, the intraoral neurostimulation device further includes a second electrode pair comprising a second reference electrode and a second stimulating electrode. The second stimulating electrode is supported by the mouthpiece and is configured to be positioned between the at least one genioglossus muscle and the tongue of the user.

In certain embodiments, the mouthpiece comprises the at least one substrate including a first tab and a second tab. The first tab is integrally extending from a left side of the dental attachment, and the first stimulating electrode is mounted to a bottom of the first tab. The second tab is integrally extending from a right side of the dental attachment, and the second stimulating electrode is mounted to a bottom of the second tab.

In certain embodiments, the first stimulating electrode is attached to a left side of the mouthpiece and configured to be positioned between a left genioglossus muscle and the tongue of the user, wherein the first stimulating electrode is configured to transmit the first electrical signal to provide the non-invasive electrical stimulation to the left genioglossus muscle of the user. The second stimulating electrode is attached to a right side of the mouthpiece and configured to be positioned between a right genioglossus muscle and the tongue of the user, wherein the second stimulating electrode is configured to transmit a second electrical signal to provide the non-invasive electrical stimulation to the right genioglossus muscle of the user.

In certain embodiments, the intraoral neurostimulation device further includes a first waveform generator in electrical communication with the first electrode pair and configured to deliver a first biphasic waveform to the first electrode pair, and a second waveform generator in electrical communication with the second electrode pair and configured to deliver a second biphasic waveform to the second electrode pair.

In certain embodiments, the first electrical signal includes a duration of up to 2 seconds, a pulse width of up to 500 microseconds, a frequency in a range between 20 and 200 Hz, and an amplitude in a range up to 5 mA.

In certain embodiments, an intraoral neurostimulation system includes the intraoral neurostimulation device and a respiratory sensor configured to generate a breathing monitoring signal indicative of a breathing characteristic of the user.

In certain embodiments, the breathing characteristic indicates cessation of breathing for a predetermined time period indicative of an apneic episode, and the intraoral neurostimulation device is configured to transmit the first electrical signal responsive to the breathing monitoring signal to draw the tongue of the user forward within the mouth of the user.

In certain embodiments, the breathing characteristic comprises a breathing pattern, and the intraoral neurostimulation device is configured to transmit a plurality of first electrical signals responsive to the breathing monitoring signal to synchronize the plurality of first electrical signals with the breathing pattern of the user.

In certain embodiments, the respiratory sensor comprises an intraoral respiratory sensor.

In certain embodiments, the respiratory sensor includes an external respiratory sensor configured to mount to a body of the user.

In certain embodiments, an intraoral neurostimulation system includes the intraoral neurostimulation device and a respiratory sensor. The intraoral neurostimulation device further includes a first waveform generator in electrical communication with the first electrode pair, and a battery in electrical communication with the first waveform generator. The respiratory sensor is configured to generate a breathing monitoring signal indicative of a breathing characteristic of the user. The first waveform generator is in communication with the respiratory sensor, and is configured to transmit the first electrical signal to the first stimulating electrode responsive to the breathing monitoring signal.

In another aspect, the disclosure relates to a method of using an intraoral neurostimulation device for treating or ameliorating effects of sleep apnea. The method includes generating, by a respiratory sensor, a respiratory signal indicative of a breathing characteristic of a user. The method further includes electronically processing the respiratory signal to determine whether the breathing characteristic is indicative of an apneic episode. The method further includes transmitting, by a first waveform generator, a first electrical signal to a first stimulating electrode of a first electrode pair of the intraoral neurostimulation device. The first stimulating electrode is supported by a mouthpiece within a mouth of the user and positioned between at least one genioglossus muscle and a tongue of the user. The method further includes transmitting, by the first stimulating electrode, the first electrical signal to provide non-invasive electrical stimulation to the at least one genioglossus muscle of the user.

In certain embodiments, the method further includes transmitting, by a second waveform generator, a second electrical signal to a second stimulating electrode of a second electrode pair of the intraoral neurostimulation device. The second stimulating electrode is supported by the mouthpiece and positioned between the at least one genioglossus muscle and the tongue of the user. The method further includes transmitting, by the second stimulating electrode, the second electrical signal to provide the non-invasive electrical stimulation to the at least one genioglossus muscle of the user.

In certain embodiments, the first electrical signal is transmitted for a duration of up to 2 seconds with a pulse width of up to 500 microseconds, a frequency in a range between 20 and 200 Hz, and an amplitude in a range up to 5 mA.

In another aspect, any one or more aspects or features described herein may be combined with any one or more other aspects or features for an additional advantage.

Other aspects and embodiments will be apparent from the detailed description and accompanying drawings.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWINGS

FIG. 1 is a block diagram illustrating an intraoral neurostimulation system for obstructive sleep apnea (OSA), with the intraoral neurostimulation system including an intraoral neurostimulation device.

FIG. 2 is a side elevational view of a stimulating electrode and a substrate of the intraoral neurostimulation device of FIG. 1 positioned between a tongue and a genioglossus muscle of a user.

FIG. 3A is a perspective view of an embodiment of the intraoral neurostimulation device for OSA including an external device.

FIG. 3B is a top plan view of the intraoral neurostimulation device of FIG. 3A mounted within a mouth of a user.

FIG. 4A is a perspective view of an embodiment of the intraoral neurostimulation device for OSA configured to be entirely positioned within a mouth of a user.

FIG. 4B is a top plan view of the intraoral neurostimulation device of FIG. 4A mounted within a mouth of a user.

FIG. 5 is a schematic diagram illustrating a flow of power and information signals throughout the intraoral neurostimulation device.

FIG. 6 is a flowchart identifying steps of a method for using the intraoral neurostimulation device of FIG. 1.

FIG. 7 is a schematic diagram of one embodiment of a computer system for use with an intraoral neurostimulation device as disclosed herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Disclosed herein is a non-invasive intraoral neurostimulation device to treat obstructive sleep apnea (OSA) while improving adherence, effectiveness, and outcomes. The intraoral neurostimulation device includes a mouthpiece, at least one electrode pair having a reference electrode and a stimulating electrode supported by the mouthpiece, and a substrate positioned between a tongue of a user and the stimulating electrode. The intraoral neurostimulation device is configured to deliver non-invasive electrical neurostimulation to the motor neurons of at least one genioglossus muscle to induce a muscle contraction (i.e., a temporary increase in muscle tone) of the tongue, thereby eliminating the blockage in the upper airway and returning normal airflow to the user. In certain embodiments, the intraoral neurostimulation device is paired with a respiratory sensor to deliver stimulation only during apneic episodes, reducing the total amount of stimulation time (e.g., to an estimated 1-4 minutes per night for mild to severe OSA cases).

In certain embodiments, the intraoral neurostimulation device is customizable for comfort (e.g., functions painlessly, functions quietly, safe, painless, does not cause itching, keeps mouth moist, etc.), durable (e.g., survives drops), affordable (e.g., low cost), reusable (e.g., treatment lasts more than one night of use, suitable for cleaning after each use), and/or reliable, etc. Further, in certain embodiments, the intraoral neurostimulation device is simple to set up, use, and maintain for non-invasive treatment. For example, in certain embodiments, the intraoral neurostimulation device is easy to set up (e.g., easy device placement, sticks securely to skin), easy to use (simple directions), easy to maintain (e.g., easy cleaning, easily replaceable parts), portable (e.g., inconspicuous, small), convenient (e.g., suitable for daily use, short adjustment period, remains dry), has customizable settings (e.g., programmable by users, has self-adjustment settings), reliable (e.g., opens airway for breathing, overpowers or bypasses nose congestion, functions during power outages, detects sleep and wake periods, prevents air leakage), etc. In certain embodiments, the intraoral neurostimulation device sends feedback to users and/or physicians (e.g., provides feedback to patients, collects and analyzes data and generates reports, sends generated reports to patients and/or physicians).

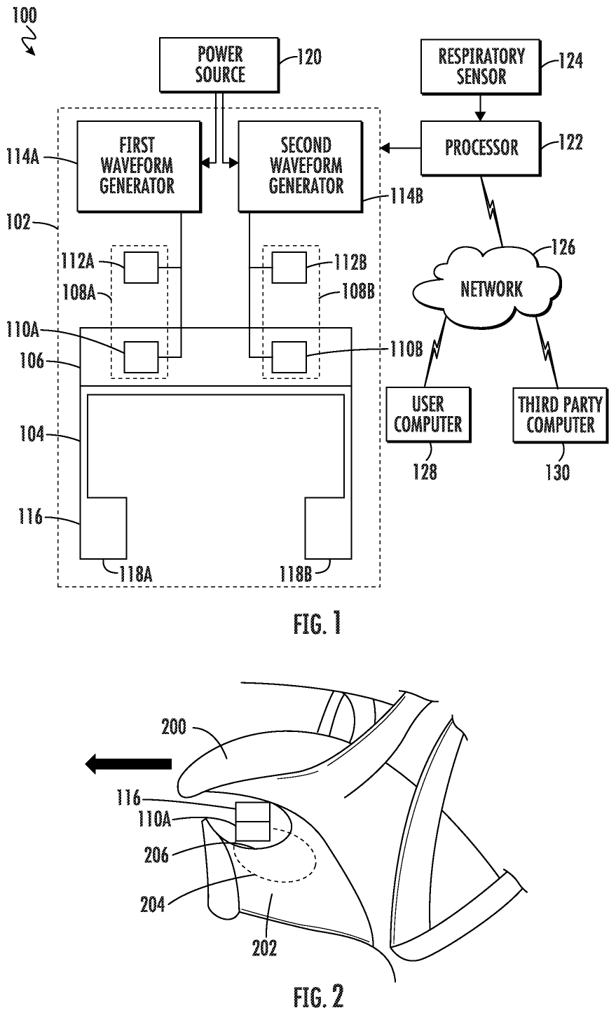

FIG. 1 is a block diagram illustrating an intraoral neurostimulation system 100 for OSA, the intraoral neurostimulation system 100 including an intraoral neurostimulation device 102. The intraoral neurostimulation device 102 includes a mouthpiece 104 and at least one substrate 106 attached to the mouthpiece 104. The intraoral neurostimulation device 102 further includes a first electrode pair 108A (including a first stimulating electrode 110A and a first reference electrode 112A) and a second electrode pair 1086 (including a second stimulating electrode 1106 and a second reference electrode 112B) to intraorally stimulate a mouth of a user. The intraoral neurostimulation device 102 includes a first waveform generator 114A to transmit a first electrical signal to the first electrode pair 108A and a second waveform generator 1146 to transmit a second electrical signal to the second electrode pair 108B. In certain embodiments, a single waveform generator is in electrical communication with the first electrode pair 108A and the second electrode pair 108B.

In certain embodiments, the mouthpiece 104 is an intraoral mouthpiece made of biocompatible material that engages at least one tooth of the lower jaw of the user to hold the first and second stimulating electrodes 110A, 1106 (and/or the first and second reference electrodes 112A, 112B) in place within the mouth. In certain embodiments, the mouthpiece 104 includes DentaPro 2000 dental-grade silicone mouth guard, stainless steel 304 hex screw cap, and/or polyolefin shrink tubing. In certain embodiments, the mouthpiece 104 houses electronic components (e.g., the first waveform generator 114A and/or the second waveform generator 114B) and secures the first and second stimulating electrodes 110A, 1106 (and/or first and second reference electrodes 112A, 112B) in the proper placement within the mouth for stimulation. The mouthpiece 104 includes a dental attachment 116 configured to be removably received by at least one tooth (e.g., at least one bottom tooth) within the mouth of the user. For example, the dental attachment 116 includes a left dental attachment portion 118A configured to be removably received by at least one left tooth (e.g., at least one left bottom tooth) and a right dental attachment portion 1186 configured to be removably received by at least one right tooth (e.g., at least one right bottom tooth). In certain embodiments, the dental attachment 116 is configured to be removably received by all of the bottom teeth. In other words, the mouthpiece 104 could either cover all of the teeth (e.g., like a mouthguard), or only cover the back molars with the front incisors remaining uncovered. In certain embodiments, the mouthpiece 104 includes the at least one substrate 106 for mounting the first and second stimulating electrodes 110A, 1106 of the first electrode pair 108A and the second electrode pair 108B. In other embodiments, the first and second stimulating electrodes 110A, 1106 may be mounted to the mouthpiece 104 and the at least one substrate 106 applied separately to the first and second stimulating electrodes 110A, 110B.

The first and second stimulating electrodes 110A, 1106 deliver non-invasive electrical stimulation to the motor neurons of the lingual muscles, thereby inducing a muscle contraction (e.g., a temporary increase in muscle tone). It is noted that only one first electrode pair 108A may be used, although two stimulating electrodes 110A, 110B provide a tighter range of settings across various users. The first electrode pair 108A includes a first stimulating electrode 110A attached to the mouthpiece 104, and the second electrode pair 108B includes a second stimulating electrode 1106 attached to the mouthpiece 104. In certain embodiments, the first stimulating electrode 110A and/or the second stimulating electrode 110B are made of biocompatible materials (e.g., stainless steel).

In certain embodiments, the intraoral neurostimulation device 102 mouthpiece 104 is molded to the patient's lower teeth and contains a metal wire for connecting the various electrical components. The electrodes 110A, 1106, 112A, 1126 are attached to the wire and draw current therefrom. The electrodes 110A, 110, 112A, 112B sit inferior and anterior to the base of the tongue, so as to stimulate the motor neurons that innervate the genioglossus muscle, thereby producing contraction and bringing the tongue forward. In certain embodiments, these electrodes can be manufactured by simply connecting a metal (or metal-containing) lead (silver, platinum, mixed-metal oxide (MMO), titanium, etc.) to an inert material such as graphite or silicone that contains the lead to insulate the lead and control contact points. In certain embodiments, the first reference electrode 112A and/or the second reference electrode 112B are made of biocompatible materials (e.g., stainless steel).

FIG. 2 is a side elevational view of the first stimulating electrode 110A and the substrate 106 of the intraoral neurostimulation device 102 of FIG. 1 positioned between a tongue 200 and genioglossus muscle 202 of a user. The genioglossus muscle 202 is the fleshy part of the mouth located under the tongue 200 (i.e., not in the tongue itself). The intraoral neurostimulation device 102 is configured to target a targeted region 204 of the genioglossus muscle 202 located below the tongue 200. It is noted that although the first stimulating electrode 110A and the first reference electrode 112A of the first electrode pair 108A are discussed, the same also apply to the second stimulating electrode 110B and the second reference electrode 112B of the second electrode pair 108B. Mounting the first stimulating electrode 110A (and/or the first reference electrode 112A) to the mouthpiece 104 provides consistent electrode placement within a mouth of a user (thereby reducing any potential signal noise factor). To determine the optimal location for electrode placement, a picture may be taken of the mouth of the user and a graph overlaid onto the picture. Electrodes may be arranged to stimulate each marked position by cross-checking with the picture to ensure consistency.

The genioglossus muscle 202 has the ability to produce a force of anywhere between 5-28 N. Since the intraoral neurostimulation device 102 is attempting to simply erect the tongue 200 without drastically moving the entirety of the tongue 200 in a lateral motion, the contractile force that will be necessary is closer to 5-12 N. Using the rate of contraction, the force of contraction, and the total number of myosin heads that are essentially causing the contraction, the overall length change that will warrant a complete contraction event can be determined. This will determine if the stimulation by the first and second stimulating electrodes 110A, 110B is adequately stimulating the genioglossus muscle 202 to produce a contraction event. Accordingly, this information can also be used to determine the appropriate amount of stimulation needed to initiate a contraction event. Using electromyography (EMG) evaluation, the specific contractile force of the genioglossus muscle 202 can be detected and compared to the theoretical value. Using the comparison, the stimulation efficacy can be evaluated to determine whether the tongue 200 was stimulated enough for contraction, causing adequate movement and resulting in an unblockage of the airway.

Referring to FIG. 1 and FIG. 2, the first stimulating electrode 110A and the second stimulating electrode 110B are configured to face downwards to generate an electric field that encompasses motor neurons that innervate the genioglossus muscle 202 to contract the genioglossus muscle 202 and pull the tongue 200 forward and open the airway for breathing. The first and second stimulating electrodes 110A, 110B are directly in contact with the sublingual floor 206, with the first stimulating electrode 110A targeting the left genioglossus muscle and the right stimulating electrode 110B targeting the right genioglossus muscle.

In certain embodiments, utilizing a two-electrode configuration allows simultaneous stimulation of both left and right genioglossus muscles 202, inducing forward movement of the tongue 200. In certain embodiments, placement of the first and second stimulating electrodes 110A, 110B may depend upon fitting of the intraoral neurostimulation device 102 for the individual user. The first stimulating electrode 110A is supported by the mouthpiece 104 and is configured to be positioned between at least one genioglossus muscle 202 and a tongue 200 of the user. The at least one substrate 106 is arranged between the first stimulating electrode 110A and the tongue 200 of the user. The first stimulating electrode 110A is configured to transmit a first electrical signal to provide non-invasive electrical stimulation to the at least one genioglossus muscle 202 of the user. The substrate 106 insulates the tongue 200 from the first electrical signal. Thus, the substrate 106 better directs the electrical stimulation to the targeted region 204. In certain embodiments, the first stimulating electrode 110A is attached to a left side of the mouthpiece 104 and configured to be positioned between a left genioglossus muscle 202 and the tongue 200 of the user. The first stimulating electrode 110A is configured to transmit the first electrical signal to provide the non-invasive electrical stimulation to the left genioglossus muscle 202 of the user.

Similarly, the second stimulating electrode 110B is supported by the mouthpiece 104 and is configured to be positioned between at least one genioglossus muscle 202 and a tongue 200 of the user. The at least one substrate 106 is arranged between the second stimulating electrode 110B and the tongue 200 of the user. The second stimulating electrode 110B is configured to transmit a second electrical signal to provide non-invasive electrical stimulation to the at least one genioglossus muscle 202 of the user. The substrate 106 insulates the tongue 200 from the second electrical signal. Thus, the substrate 106 better directs the electrical stimulation to the targeted region 204. In certain embodiments, the second stimulating electrode 110B is attached to a right side of the mouthpiece 104 and configured to be positioned between a right genioglossus muscle 202 and the tongue 200 of the user. The second stimulating electrode 110B is configured to transmit a second electrical signal to provide the non-invasive electrical stimulation to the right genioglossus muscle 202 of the user.

The first and second reference electrodes 112A, 112B (of which each may also be referred to herein as a grounding electrode or generally herein as reference electrode 112) can be placed intraorally or externally on the skin. In certain embodiments, the intraoral neurostimulation device 102 uses a reference electrode 112 that could be body-mounted (e.g., on the clavicle). In certain embodiments, the reference electrode 112 is placed intraorally and is configured to be in conductive electrical communication with a jaw of the user within the mouth of the user. In certain embodiments, this reference electrode 112 would be placed at the back of the mouth connected to the mouthpiece 104 and contact a bony part of the mouth (e.g., the molars). In certain embodiments, the reference electrode 112 is configured to be in conductive electrical communication with skin of the user.

Referring back to FIG. 1, in certain embodiments, the intraoral neurostimulation device 102 includes at least one waveform generator, such as the first waveform generator 114A and the second waveform generator 1146 (referred to generally as waveform generator 114) to deliver an electrical signal to the first stimulating electrode 110A of the first electrode pair 108A and/or the second stimulating electrode 110B of the second electrode pair 108B. The waveform delivered to the user is safe, comfortable, and effective. In certain embodiments, the waveform generator 114 utilizes two potentiometers to change frequency and amplitude of the generated pulse allowing user customization. In certain embodiments, the intraoral neurostimulation device 102 is connected to the square wave output of a waveform generator 114. In certain embodiments, an adjustable waveform generator 114 is used to allow for variation in pulse frequency and amplitude. In certain embodiments, a waveform could be applied to the hand to ensure a comfortable and effective stimulation, which can then be transferred to the mouth.

In certain embodiments, the intraoral neurostimulation device 102 includes a first waveform generator 114A in electrical communication with the first electrode pair 108A and configured to deliver a first biphasic waveform to the first electrode pair 108A, and/or a second waveform generator 114B in electrical communication with the second electrode pair 108B and configured to deliver a second biphasic waveform to the second electrode pair 108B. A variety of waveform parameters may be used. For example, in certain embodiments, the first electrical signal includes a duration of up to 2 seconds, a pulse width of up to 500 microseconds (e.g., about 150 microseconds, 100 microseconds, etc.), a frequency in a range between 20 and 200 Hz (e.g., between 80 and 100 Hz), and/or an amplitude in a range up to 5 mA (e.g., between 2 to 2.25 mA). In certain embodiments, the electrical signal includes an amplitude up to 5 mA, a pulse width of up to 500 microseconds (e.g., up to 150 microseconds and/or about 100 microseconds), a frequency between 0-100 Hz, and/or a pulse train duration of 1-3 s (e.g., based on severity of OSA and breathing frequency). In certain embodiments, the mode is synchronous with a pulse width of up to 150 microseconds (e.g., about 100 microseconds), ramp 1 s, on 3 s, off 1 s. In certain embodiments, the frequency is 90 Hz at 2 mA. In other embodiments, the frequency is between 80 and 100 Hz at 2.125 mA. A low pulse width (e.g., less than 150 microseconds) means the user likely will not be able to feel the stimulation on their mouth tissue, which is important to prevent the user from waking up from sleeping. This parameter may be customizable by the user, as some people may be more sensitive to certain pulse settings over others.

It is noted that an output voltage needed in a given current range to reach the nerve of the genioglossus muscle 202 can be theoretically calculated based on a resistance of the intraoral neurostimulation system 100. For example, such calculations may include resistance and capacitance of double layer electrodes, resistance of saliva (between electrode and oral mucosa tissue), resistance and capacitance of oral mucosa tissue (parallel configuration), resistance of lamina, resistance of genioglossus muscle(s), frequency waveform, etc. Calculations may include determining how much current is being lost in the tissue and not reaching the nerve for modulation. The resistance and capacitance values of each physiological layer can be looked up or can be predicted (e.g., extrapolated) with assumptions from similar anatomic parts from other areas of the body. For example, the oral mucosa layer is very similar to the stratum corneum and a similar resistance can be assumed.

As an example, by utilizing this model and with assumptions made for values of impedance and capacitance, the total impedance of the circuit is 490.9Ω. However, the final impedance does not consider the impedance that the current encounters on its way out of the body towards the reference electrode. The model assumes that a similar amount of impedance will be encountered on the way out as on the way in. For the current amplitude range to be 0.5-5 mA, the voltage required from the electrode is in a range of 0.491 V-4.91 V, which falls within the target specification of 0-5 V for output voltage.

A range of stimulation settings may be used to evoke the desired motoneuron response in the genioglossus muscle 202. One exemplary model for the stimulation settings includes equations that model electrical stimulation on human skin. Because each patient will require different levels of stimulation, it is not possible to solve for a single value for each unknown variable, as this will be unique to each person. For example, in certain embodiments, the current can be 0-100 mA, and time can be 50-1000 microseconds, such that voltage is 0-2 V and/or 5-14 V.

Referring back to FIG. 1, in certain embodiments, the power source 120 includes a battery (e.g., a lithium-ion battery) housed in the mouthpiece 104. In this configuration, a wireless charger could be used to charge the battery without compromising any hermetic seal. Alternatively, the battery could be housed in body-mounted (extraoral) components. In this configuration, a wired connection to the intraoral first and second stimulating electrodes 110A, 110B may be used.

It is noted that a power for a rechargeable battery to produce sufficient power and charge/discharge time can be theoretically calculated. For example, such calculations may include charge/discharge rate from open source voltage, discharge current, internal resistance, rated capacity, and charge/discharge efficiency (which depends on various environmental factors such as temperature, charge-discharge rate, battery aging, etc.).

In certain embodiments, the intraoral neurostimulation system 100 includes a processor 122 (which may be referred to herein as a controller) and/or a respiratory sensor 124 (which may be referred to herein as a breathing sensor). The processor 122 and/or the respiratory sensor 124 may be housed intraorally (e.g., within the mouthpiece 104) and/or external to the body (e.g., mounted to the body).

The processor 122 is configured to control delivery of stimulation by the waveform generators 114 based on the respiratory sensor 124. The processor 122 is configured to determine when to stimulate during the breathing cycle based on the respiratory sensor 124.

The respiratory sensor 124 monitors and records the user's breathing. The respiratory sensor 124 is configured to generate a breathing monitoring signal indicative of a breathing characteristic of the user. The first waveform generator 114A is in communication with the respiratory sensor 124. The first waveform generator 114A is configured to transmit the first electrical signal to the first stimulating electrode 110A responsive to the breathing monitoring signal. The battery is in electrical communication with the first waveform generator 114A. In certain embodiments, the breathing characteristic indicates cessation of breathing for a predetermined time period indicative of an apneic episode, and the intraoral neurostimulation device 102 is configured to transmit the first electrical signal responsive to the breathing monitoring signal to draw the tongue 200 of the user forward within the mouth of the user.

In certain embodiments, the breathing characteristic comprises a breathing pattern, and the intraoral neurostimulation device 102 is configured to transmit a plurality of first electrical signals responsive to the breathing monitoring signal to synchronize the plurality of first electrical signals with the breathing pattern of the user. In certain embodiments, the respiratory sensor comprises an intraoral respiratory sensor. In certain embodiments, the respiratory sensor 124 includes an external respiratory sensor configured to mount to a body of the user.

When the respiratory sensor 124 detects that the user has stopped breathing, indicating an apneic episode, the intraoral neurostimulation device 102 delivers stimulation (e.g., for a maximum of two seconds). The intraoral neurostimulation device 102 only stimulates during specific periods in the breathing cycle to reduce total stimulation time throughout the night. The specific timing of the stimulation could include intraoral neurostimulation (stimulation pulse trains) when an apneic episode is detected (cessation of breathing) and/or intraoral neurostimulation upon the initialization of each breath. Stimulation pulses are interleaved (i.e., the pulses in the pulse train to one channel are spaced in between pulses to the other channel) and the pulse trains can be up to a few seconds in duration. The intraoral neurostimulation device 102 may be configured to regulate the rate of stimulation, such as to stimulate on every inhale, stimulate only during apneic episodes, etc. In certain embodiments, this reduces the total amount of stimulation time to an estimated 1-4 minutes per night for extreme cases of OSA.

In certain embodiments, the time in between breaths is recorded and the intraoral neurostimulation device 102 only stimulates when there is a sufficiently long gap between breaths. This stimulation needs higher amplitude to produce an effective contraction, but may be easier to implement in software. In certain embodiments, the model incorporates recorded breathing responses as a function of stimulation timing and pairs the function with a breathing pattern found in OSA patients.

In certain embodiments, a breathing sensor may be built that includes a strap knitted out of yarn and conductive thread, which is designed to detect breaths in response to a change in length (due to stretching) when the user inhales. In certain embodiments, the breathing sensor incorporates a rubber stretch sensor hooked onto an inelastic strap, such that all of the stretch is felt by the stretch sensor. The body-mounted chest band breathing sensor utilizes stretch sensors to quantify resistance.

In certain embodiments, the intraoral neurostimulation system 100 stores and sends data to generate sleep reports overnight and/or send sleep reports to users and/or physicians. For example, in certain embodiments, the processor 122 communicates over a network 126 to a user's computer 128 and/or a third party computer 130 (e.g., a physician's computer).

In certain embodiments, the intraoral neurostimulation system 100 provides a stimulation intensity (e.g., up to 5 mA, 1.75-2.25 mA, etc.) to produce sufficient current to induce stimulation, a low pulse width (e.g., up to 500 microseconds, up to 150 microseconds, about 100 microseconds, etc.) to be painless, and a pulse duration (e.g., 3 s) to produce minimal side effects of stimulation. In certain embodiments, the biocompatible material (e.g., silicone, stainless steel) is safe, the product size (e.g., 2 in.×2 in.×0.5 in.) is minimally intrusive, the product weight (e.g., 20-50 g) is comfortable, and the battery power (0-14 V) is portable with wireless charging (e.g., magnetic charging) and low manufacturing cost (e.g., around $45).

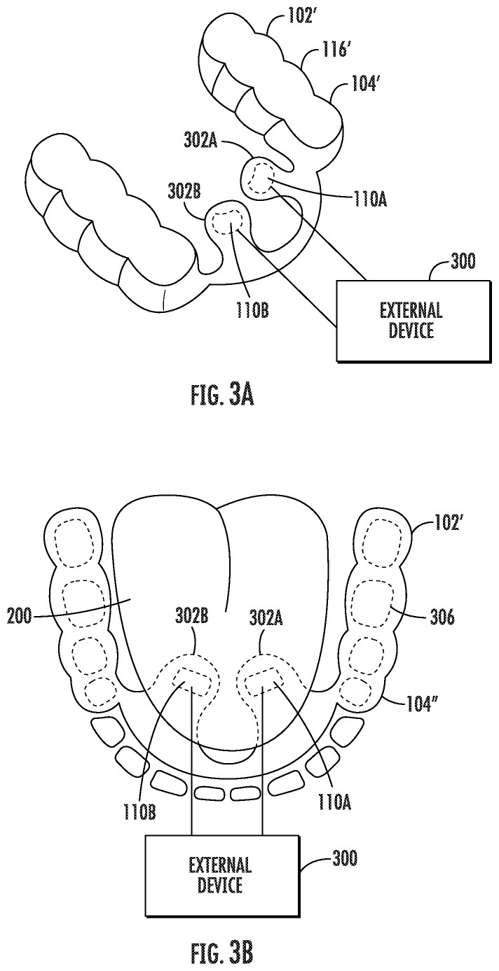

FIGS. 3A and 3B are perspective and top plan views, respectively, of an embodiment of an intraoral neurostimulation device 102′ for OSA including an external device 300. In certain embodiments, the mouthpiece 104′ includes the dental attachment 116′ and the at least one substrate 106 embodied as a first tab 302A and a second tab 302B. The first tab 302A integrally extends from a left side of the dental attachment 116′, and a second tab 302B integrally extends from a right side of the dental attachment 116′. The first stimulating electrode 110A is mounted to a bottom of the first tab 302A. The second stimulating electrode 110B is mounted to a bottom of the second tab 302B. The first stimulating electrode 110A and the second stimulating electrode 110B are in electrical communication with the external device 300 which includes, for example, the waveform generator 114 and/or any other feature described in FIG. 1. The intraoral neurostimulation device 102′ is configured for placement inside the mouth with the first and second stimulating electrodes 110A, 110B positioned below the tongue 200. The mouthpiece 104′ is fitted to back teeth 306 to anchor the intraoral neurostimulation device 102′ and keep the first and second stimulating electrodes 110A, 110B in place.

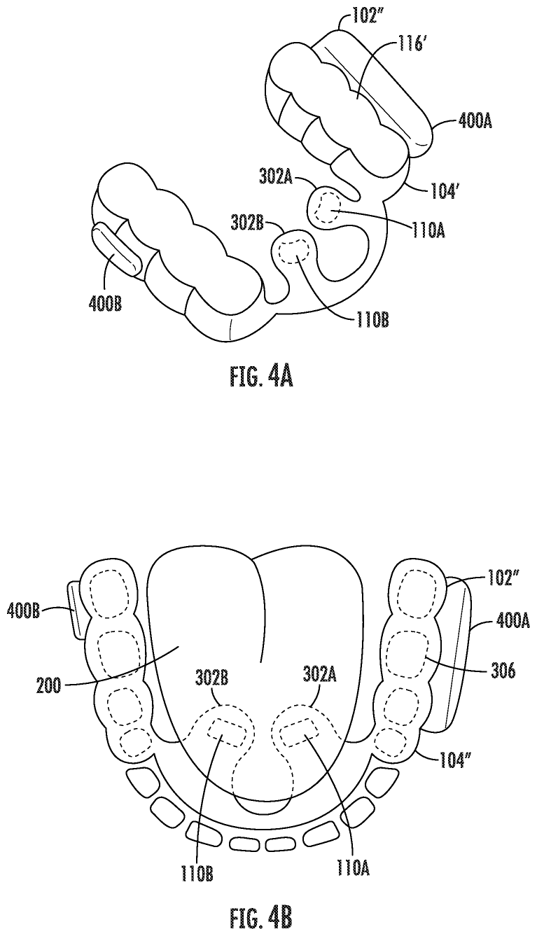

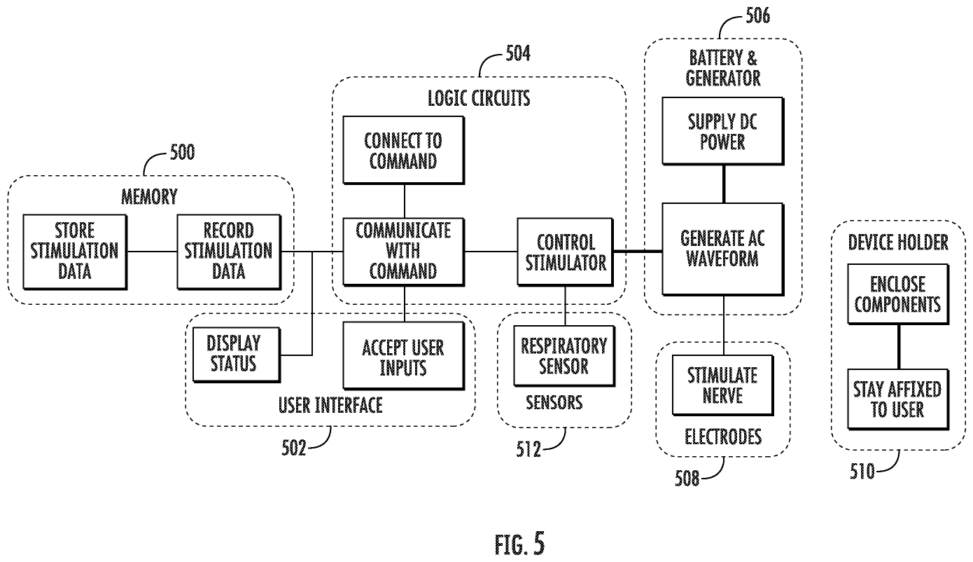

FIGS. 4A and 4B are perspective and top plan views, respectively, of an embodiment of an intraoral neurostimulation device 102″ for OSA configured to be entirely positioned within the mouth of the user. As similarly noted above, in certain embodiments, a mouthpiece 104″ includes the dental attachment 116′ and the at least one substrate 106 embodied as the first tab 302A and the second tab 302B. The first tab 302A integrally extends from a left side of the dental attachment 116′, and the second tab 302B integrally extends from a right side of the dental attachment 116′. The first stimulating electrode 110A is mounted to a bottom of the first tab 302A. The second stimulating electrode 110B is mounted to a bottom of the second tab 302B. The intraoral neurostimulation device 102″ is configured for placement inside the mouth with the first and second stimulating electrodes 110A, 110B positioned below the tongue 200. The mouthpiece 104″ is fitted to the back teeth 306 to anchor the intraoral neurostimulation device 102″ and keep the first and second stimulating electrodes 110A, 110B in place.

In certain embodiments, the intraoral neurostimulation device 102″ includes the mouthpiece 104″, the waveform generator 114, and the breathing sensor 124 (see FIG. 1). In this embodiment, the neurostimulation device 102″ includes a first housing unit 400A on the posterior left hand side of the intraoral neurostimulation device 102″ for the breathing sensor 124, the power source 120, the waveform generator 114 (see FIG. 1), and a second housing unit 400B on the posterior right hand side of the intraoral neurostimulation device 102″ for the first and second reference electrodes 112A, 112B (see FIG. 1). In certain embodiments, the first housing unit 400A may also house the processor 122 (e.g., logic circuits) and/or memory.

FIG. 5 is a schematic diagram illustrating a flow of power and information signals throughout the intraoral neurostimulation device 102. In certain embodiments, the intraoral neurostimulation device 102 includes a memory 500, a user interface 502, logic circuits 504, a battery and generator 506, electrodes 508, a device holder 510, and sensors 512. The memory 500 stores stimulation data and records stimulation data. The user interface 502 displays status of the intraoral neurostimulation device 102 and accepts user inputs. The logic circuits 504 connect to command (e.g., processor), communicate with command, receive inputs from the stimulation data and user inputs, as well as controls the display status. Logic circuits 504 control the control stimulator, which directs operation of the waveform. The battery and generator 506 supply DC power and an AC waveform is generated to stimulate nerves by the electrodes 508. The device holder 510 encloses components and stays affixed to the user. The sensors 512 (e.g., respiratory sensor) can communicate with the control simulator of the logic circuits 504 (e.g., via analog digital converter (ADC), Bluetooth, etc.).

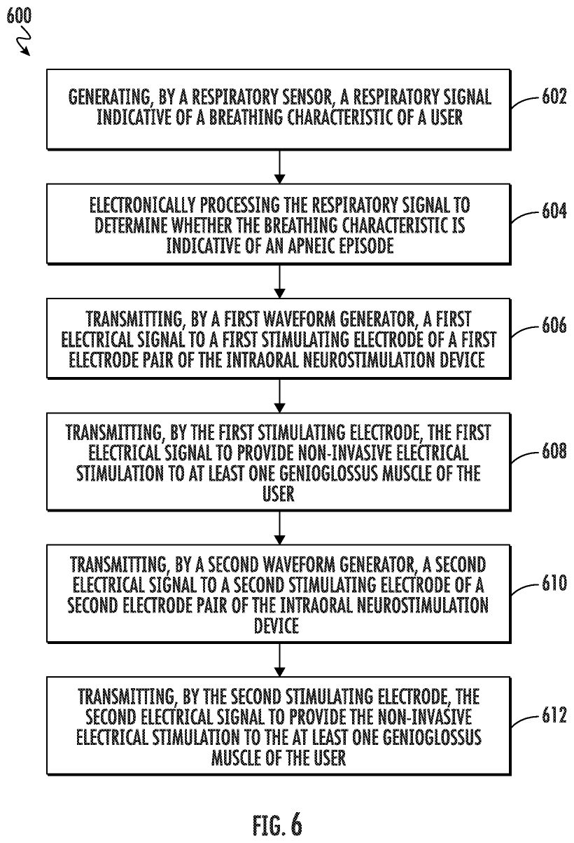

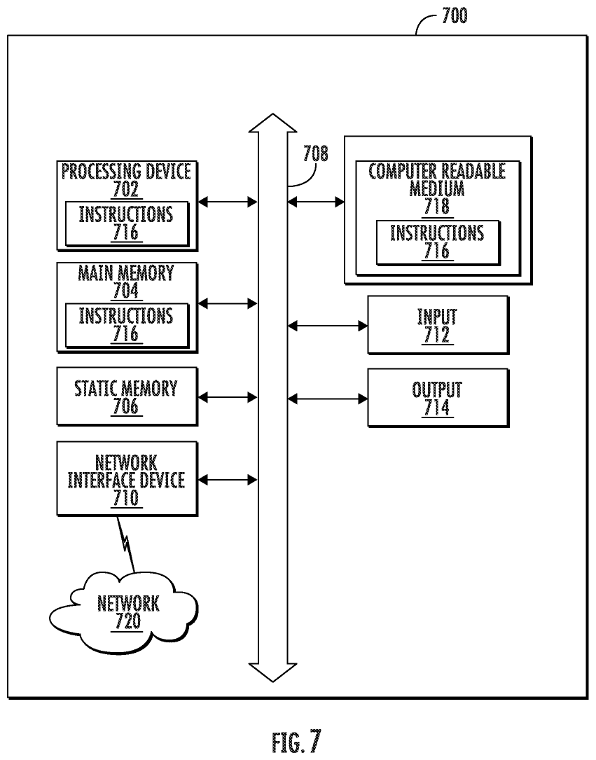

FIG. 6 is a flowchart 600 identifying steps of a method for using the intraoral neurostimulation device 102 of FIG. 1. Step 602 includes generating, by a respiratory sensor 124, a respiratory signal indicative of a breathing characteristic of a user. Step 604 includes electronically processing the respiratory signal to determine whether the breathing characteristic is indicative of an apneic episode.

Step 606 includes transmitting, by a first waveform generator 114A, a first electrical signal to a first stimulating electrode 110A of a first electrode pair 108A of the intraoral neurostimulation device 102. The first stimulating electrode 110A is supported by a mouthpiece 104 within a mouth of the user and positioned between at least one genioglossus muscle 202 and a tongue 200 of the user. Step 608 includes transmitting, by the first stimulating electrode 110A, the first electrical signal to provide non-invasive electrical stimulation to the at least one genioglossus muscle 202 of the user.

Step 610 includes transmitting, by a second waveform generator 114B, a second electrical signal to a second stimulating electrode 110B of a second electrode pair 108B of the intraoral neurostimulation device 102. The second stimulating electrode 110B is supported by the mouthpiece 104 and is positioned between the at least one genioglossus muscle 202 and the tongue 200 of the user. Step 612 includes transmitting, by the second stimulating electrode 110B, the second electrical signal to provide the non-invasive electrical stimulation to the at least one genioglossus muscle 202 of the user.

In certain embodiments, the first electrical signal is transmitted for a duration of up to 2 seconds with a pulse width of up to 500 microseconds (e.g., up to 150 microseconds, about 100 microseconds, etc.), a frequency in a range between 20 and 200 Hz (e.g., between 80 and 100 Hz), and an amplitude in a range up to 5 mA (e.g., between 2 to 2.25 mA).

FIG. 7 is a schematic diagram of one embodiment of a computer system 700 that can be included in any component of the systems or methods disclosed herein. In this regard, the computer system 700 is adapted to execute instructions from a computer-readable medium to perform these and/or any of the functions or processing described herein.

In this regard, the computer system 700 in FIG. 7 may include a set of instructions that may be executed to program and configure programmable digital signal processing circuits for supporting scaling of supported communications services. The computer system 700 may be connected (e.g., networked) to other machines in a local area network (LAN), an intranet, an extranet, or the Internet. While only a single device is illustrated, the term “device” shall also be taken to include any collection of devices that individually or jointly execute a set (or multiple sets) of instructions to perform any one or more of the methodologies discussed herein. The computer system 700 may be a circuit or circuits included in an electronic board card, such as a printed circuit board (PCB), a server, a personal computer, a desktop computer, a laptop computer, a personal digital assistant (PDA), a computing pad, a mobile device, or any other device, and may represent, for example, a server or a user's computer.

The computer system 700 in this embodiment includes a processing device or processor 702, a main memory 704 (e.g., read-only memory (ROM), flash memory, dynamic random access memory (DRAM), such as synchronous DRAM (SDRAM), etc.), and a static memory 706 (e.g., flash memory, static random access memory (SRAM), etc.), which may communicate with each other via a data bus 708. Alternatively, the processing device 702 may be connected to the main memory 704 and/or static memory 706 directly or via some other connectivity means. The processing device 702 may be a controller, and the main memory 704 or static memory 706 may be any type of memory.

The processing device 702 represents one or more general-purpose processing devices, such as a microprocessor, central processing unit, or the like. More particularly, the processing device 702 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 702 is configured to execute processing logic in instructions for performing the operations and steps discussed herein.

The computer system 700 may further include a network interface device 710. The computer system 700 also may or may not include an input 712, configured to receive input and selections to be communicated to the computer system 700 when executing instructions. The computer system 700 also may or may not include an output 714, including but not limited to a display, a video display unit (e.g., a liquid crystal display (LCD) or a cathode ray tube (CRT)), an alphanumeric input device (e.g., a keyboard), and/or a cursor control device (e.g., a mouse).

The computer system 700 may or may not include a data storage device that includes instructions 716 stored in a computer readable medium 718. The instructions 716 may also reside, completely or at least partially, within the main memory 704 and/or within the processing device 702 during execution thereof by the computer system 700, the main memory 704 and the processing device 702 also constituting computer readable media. The instructions 716 may further be transmitted or received over a network 720 via the network interface device 710.

While the computer readable medium 718 is shown in an embodiment to be a single medium, the term “computer-readable medium” should be taken to include a single medium or multiple media (e.g., a centralized or distributed database, and/or associated caches and servers) that store the one or more sets of instructions 716. The term “computer readable medium” shall also be taken to include any medium that is capable of storing, encoding, or carrying a set of instructions for execution by the processing device and that cause the processing device to perform any one or more of the methodologies of the embodiments disclosed herein. The term “computer readable medium” shall accordingly be taken to include, but not be limited to, solid-state memories, optical medium, and magnetic medium.

The embodiments disclosed herein include various steps. The steps of the embodiments disclosed herein may be formed by hardware components or may be embodied in machine-executable instructions, which may be used to cause a general-purpose or special-purpose processor programmed with the instructions to perform the steps. Alternatively, the steps may be performed by a combination of hardware and software.

The embodiments disclosed herein may be provided as a computer program product, or software, that may include a machine-readable medium (or computer readable medium) having stored thereon instructions that may be used to program a computer system (or other electronic devices) to perform a process according to the embodiments disclosed herein. A machine-readable medium includes any mechanism for storing or transmitting information in a form readable by a machine (e.g., a computer). For example, a machine-readable medium includes: a machine-readable storage medium (e.g., ROM, random access memory (“RAM”), a magnetic disk storage medium, an optical storage medium, flash memory devices, etc.); and the like.

Unless specifically stated otherwise and as apparent from the previous discussion, it is appreciated that throughout the description, discussions utilizing terms such as “processing,” “computing,” “determining,” “displaying,” or the like, refer to the action and processes of a computer system, or a similar electronic computing device, that manipulates and transforms data and memories represented as physical (electronic) quantities within the computer system's registers into other data similarly represented as physical quantities within the computer system memories or registers or other such information storage, transmission, or display devices.

The algorithms and displays presented herein are not inherently related to any particular computer or other apparatus. Various systems may be used with programs in accordance with the teachings herein, or it may prove convenient to construct more specialized apparatuses to perform the required method steps. The required structure for a variety of these systems is disclosed in the description above. In addition, the embodiments described herein are not described with reference to any particular programming language. It will be appreciated that a variety of programming languages may be used to implement the teachings of the embodiments as described herein.

Those skilled in the art will further appreciate that the various illustrative logical blocks, modules, circuits, and algorithms described in connection with the embodiments disclosed herein may be implemented as electronic hardware, instructions stored in memory or in another computer readable medium and executed by a processor or other processing device, or combinations of both. The components of the system described herein may be employed in any circuit, hardware component, integrated circuit (IC), or IC chip, as examples. Memory disclosed herein may be any type and size of memory and may be configured to store any type of information desired. To clearly illustrate this interchangeability, various illustrative components, blocks, modules, circuits, and steps have been described above generally in terms of their functionality. How such functionality is implemented depends on the particular application, design choices, and/or design constraints imposed on the overall system. Skilled artisans may implement the described functionality in varying ways for each particular application, but such implementation decisions should not be interpreted as causing a departure from the scope of the present embodiments.

The various illustrative logical blocks, modules, and circuits described in connection with the embodiments disclosed herein may be implemented or performed with a processor, a Digital Signal Processor (DSP), an Application Specific Integrated Circuit (ASIC), a Field Programmable Gate Array (FPGA), or other programmable logic device, a discrete gate or transistor logic, discrete hardware components, or any combination thereof designed to perform the functions described herein. Furthermore, a controller may be a processor. A processor may be a microprocessor, but in the alternative, the processor may be any conventional processor, controller, microcontroller, or state machine. A processor may also be implemented as a combination of computing devices (e.g., a combination of a DSP and a microprocessor, a plurality of microprocessors, one or more microprocessors in conjunction with a DSP core, or any other such configuration).

The embodiments disclosed herein may be embodied in hardware and in instructions that are stored in hardware, and may reside, for example, in RAM, flash memory, ROM, Electrically Programmable ROM (EPROM), Electrically Erasable Programmable ROM (EEPROM), registers, a hard disk, a removable disk, a CD-ROM, or any other form of computer readable medium known in the art. A storage medium is coupled to the processor such that the processor can read information from, and write information to, the storage medium. In the alternative, the storage medium may be integral to the processor. The processor and the storage medium may reside in an ASIC. The ASIC may reside in a remote station. In the alternative, the processor and the storage medium may reside as discrete components in a remote station, base station, or server.

It is also noted that the operational steps described in any of the embodiments herein are described to provide examples and discussion. The operations described may be performed in numerous different sequences other than the illustrated sequences. Furthermore, operations described in a single operational step may actually be performed in a number of different steps. Additionally, one or more operational steps discussed in the embodiments may be combined. Those of skill in the art will also understand that information and signals may be represented using any of a variety of technologies and techniques. For example, data, instructions, commands, information, signals, bits, symbols, and chips, which may be referenced throughout the above description, may be represented by voltages, currents, electromagnetic waves, magnetic fields, particles, optical fields, or any combination thereof.

Unless otherwise expressly stated, it is in no way intended that any method set forth herein be construed as requiring that its steps be performed in a specific order. Accordingly, where a method claim does not actually recite an order to be followed by its steps, or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the invention.

Many modifications and other embodiments of the embodiments set forth herein will come to mind to one skilled in the art to which the embodiments pertain having the benefit of the teachings presented in the foregoing descriptions and the associated drawings. Therefore, it is to be understood that the description and claims are not to be limited to the specific embodiments disclosed and that modifications and other embodiments are intended to be included within the scope of the appended claims. It is intended that the embodiments cover the modifications and variations of the embodiments provided they come within the scope of the appended claims and their equivalents. Although specific terms are employed herein, they are used in a generic and descriptive sense only and not for purposes of limitation.

What is claimed is:

1. An intraoral neurostimulation device, comprising:
a mouthpiece comprising a dental attachment configured to be removably received by at least one tooth within a mouth of a user;
a first electrode pair comprising a first reference electrode and a first stimulating electrode, wherein the first stimulating electrode is supported by the mouthpiece and is configured to be positioned between at least one genioglossus muscle and a tongue of the user; and
at least one substrate arranged between the first stimulating electrode and the tongue of the user;
wherein the first stimulating electrode is configured to transmit a first electrical signal to provide non-invasive electrical stimulation to the at least one genioglossus muscle of the user.

2. The intraoral neurostimulation device of claim 1, wherein the at least one tooth comprises at least one bottom tooth of the user.

3. The intraoral neurostimulation device of claim 1, wherein the mouthpiece comprises the at least one substrate, and the at least one substrate comprises a first tab integrally extending from the dental attachment, with the first stimulating electrode being mounted to a bottom of the first tab.

4. The intraoral neurostimulation device of claim 1, wherein the first reference electrode is configured to be in conductive electrical communication with a jaw of the user via the mouth of the user.

5. The intraoral neurostimulation device of claim 1, wherein the first reference electrode is configured be in conductive electrical communication with skin of the user.

6. The intraoral neurostimulation device of claim 1, further comprising a first waveform generator in electrical communication with the first electrode pair and configured to deliver a first biphasic waveform to the first electrode pair.

7. The intraoral neurostimulation device of claim 1, wherein the intraoral neurostimulation device further comprises a second electrode pair comprising a second reference electrode and a second stimulating electrode, wherein the second stimulating electrode is supported by the mouthpiece and is configured to be positioned between the at least one genioglossus muscle and the tongue of the user.

8. The intraoral neurostimulation device of claim 7, wherein the mouthpiece comprises the at least one substrate, and the at least one substrate comprises:
a first tab integrally extending from a left side of the dental attachment, with the first stimulating electrode being mounted to a bottom of the first tab; and
a second tab integrally extending from a right side of the dental attachment, with the second stimulating electrode being mounted to a bottom of the second tab.

9. The intraoral neurostimulation device of claim 1, wherein:
the first stimulating electrode is attached to a left side of the mouthpiece and is configured to be positioned between a left genioglossus muscle and the tongue of the user, wherein the first stimulating electrode is configured to transmit the first electrical signal to provide the non-invasive electrical stimulation to the left genioglossus muscle of the user; and
the second stimulating electrode is attached to a right side of the mouthpiece and is configured to be positioned between a right genioglossus muscle and the tongue of the user, wherein the second stimulating electrode is configured to transmit a second electrical signal to provide the non-invasive electrical stimulation to the right genioglossus muscle of the user.

10. The intraoral neurostimulation device of claim 7, further comprising:
a first waveform generator in electrical communication with the first electrode pair and configured to deliver a first biphasic waveform to the first electrode pair; and
a second waveform generator in electrical communication with the second electrode pair and configured to deliver a second biphasic waveform to the second electrode pair.

11. The intraoral neurostimulation device of claim 1, wherein the first electrical signal comprises:
a duration of up to 2 seconds;
a pulse width of up to 500 microseconds;
a frequency in a range between 20 and 200 Hz; and
an amplitude up to 5 mA.

12. An intraoral neurostimulation system comprising:
the intraoral neurostimulation device of claim 1; and
a respiratory sensor configured to generate a breathing monitoring signal indicative of a breathing characteristic of the user.

13. The intraoral neurostimulation system of claim 12, wherein:
the breathing characteristic indicates cessation of breathing for a predetermined time period indicative of an apneic episode; and
the intraoral neurostimulation device is configured to transmit the first electrical signal responsive to the breathing monitoring signal to draw the tongue of the user forward within the mouth of the user.

14. The intraoral neurostimulation system of claim 12, wherein:
the breathing characteristic comprises a breathing pattern; and
the intraoral neurostimulation device is configured to transmit a plurality of electrical signals responsive to the breathing monitoring signal to synchronize the plurality of electrical signals with the breathing pattern of the user.

15. The intraoral neurostimulation system of claim 12, wherein the respiratory sensor comprises an intraoral respiratory sensor.

16. The intraoral neurostimulation system of claim 12, wherein the respiratory sensor comprises an external respiratory sensor configured to mount to a body of the user.

17. An intraoral neurostimulation system comprising:
the intraoral neurostimulation device of claim 1, further comprising a first waveform generator in electrical communication with the first electrode pair, and a battery in electrical communication with the first waveform generator; and
a respiratory sensor configured to generate a breathing monitoring signal indicative of a breathing characteristic of the user;
wherein the first waveform generator is in communication with the respiratory sensor, the first waveform generator configured to transmit the first electrical signal to the first stimulating electrode responsive to the breathing monitoring signal.

18. A method of using an intraoral neurostimulation device for treating or ameliorating effects of sleep apnea, the method comprising:
generating, by a respiratory sensor, a respiratory signal indicative of a breathing characteristic of a user;
electronically processing the respiratory signal to determine whether the breathing characteristic is indicative of an apneic episode;
transmitting, by a first waveform generator, a first electrical signal to a first stimulating electrode of a first electrode pair of the intraoral neurostimulation device, the first stimulating electrode being supported by a mouthpiece within a mouth of the user and positioned between at least one genioglossus muscle and a tongue of the user; and
transmitting, by the first stimulating electrode, the first electrical signal to provide non-invasive electrical stimulation to the at least one genioglossus muscle of the user.

19. The method of claim 18, further comprising:
transmitting, by a second waveform generator, a second electrical signal to a second stimulating electrode of a second electrode pair of the intraoral neurostimulation device, the second stimulating electrode being supported by the mouthpiece and positioned between the at least one genioglossus muscle and the tongue of the user; and
transmitting, by the second stimulating electrode, the second electrical signal to provide the non-invasive electrical stimulation to the at least one genioglossus muscle of the user.

20. The method of claim 1, wherein the first electrical signal is transmitted for a duration of up to 2 seconds with a pulse width of up to 150 microseconds, a frequency in a range between 80 and 100 Hz, and an amplitude up to 5 mA.
